(19) United States
(12) Patent Application Publication
(10) Pub. No.: US 2019/0133467 A1
(43) Pub. Date: May 9, 2019

(54) SYSTEM AND METHOD FOR IDENTIFYING BABY NEEDS

(71) Applicant: Institute For Information Industry, Taipei (TW)

(72) Inventors: Guang-Feng DENG, Taipei City (TW); Po-Chih CHEN, New Taipei City (TW); Hsiao-Hung LIN, Taipei City (TW)

(21) Appl. No.: 15/823,518

(22) Filed: Nov. 27, 2017

(30) Foreign Application Priority Data
Nov. 7, 2017 (TW) 106138450

(51) Int. Cl. A61B 5/024; A61B 5/00; A61B 5/11

(52) U.S. Cl. CPC A61B 5/02416; A61B 5/7278; A61B 2503/04; A61B 5/0077; A61B 5/1128

(57) ABSTRACT

A system and a method for identifying baby needs are disclosed. The system stores a heart rate variability (HRV) feature model comprising a relationship between HRV features and baby needs. The system receives a time-series skin image signal of a baby, and converts the time-series skin image signal into a target photoplethysmography (PPG) signal. The system also calculates a set of target HRV features according to the target PPG signal, and determines a target need of the baby according to the HRV feature model and the set of target HRV features.

### PRIORITY

This application claims priority to Taiwan Patent Application No. 106138450 filed on Nov. 7, 2017, which is hereby incorporated by reference in its entirety.

### FIELD

Embodiments of the present invention relate to an identification system and an identification method. More particularly, embodiments of the present invention relate to a system and a method for identifying baby needs.

### BACKGROUND

A baby cannot express his or her needs verbally, so the parents, caregiver or babysitters can only guess needs of the baby through the crying, facial expressions and/or actions of the baby. However, it is not correct usually. In order to solve such a problem, one kind of technology is to identify the needs of the baby by analyzing sounds of the baby, but this kind of technology cannot be used when the baby makes no sound. Besides, the baby may also make a sound even without needs. The baby may make same or similar sounds for different needs, so such a technology cannot effectively identify needs of the baby. In order to solve such a problem, another kind of technology is to identify needs of the baby by analyzing facial expressions of the baby. This technology cannot be used when the face of the baby is covered. Besides, the facial expressions of the baby are limited, and the needs of the baby are not necessarily reflected by facial expressions. Also, the baby may generate same or similar facial expressions for different needs. Thus, the technology still cannot effectively identify the needs of the baby.

Accordingly, an urgent need exists in the art to provide a more effective technology for identifying baby needs.

### SUMMARY

The disclosure includes a system for identifying baby needs. The system may comprise a storage, a transceiver and a processor electrically connected with the storage and the transceiver. The storage may be configured to store a heart rate variability (HRV) feature model that comprises a relationship between HRV features and baby needs. The transceiver may be configured to receive a time-series skin image signal of a baby. The processor may be configured to convert the time-series skin image signal into a target photoplethysmography (PPG) signal, and calculate a set of HRV features according to the target PPG signal. The processor may be further configured to identify a target need of the baby according to the HRV feature model and the set of target HRV features.

The disclosure also includes a method for identifying baby needs. The method may comprise the following steps of: receiving, by a transceiver, a time-series skin image signal of a baby; converting, by a processor, the time-series skin image signal into a target PPG signal; calculating, by the processor, a set of target HRV features according to the target PPG signal; and identifying, by the processor, a target need of the baby according to a HRV feature model stored in a storage and the set of target HRV features, wherein the HRV feature model comprises a relationship between HRV features and baby needs.

In the example embodiments disclosed herein, needs of the baby are identified by analyzing the skin image of the baby. The skin image of the baby is not limited to the face, and images of portions covered by the skin, such as the face, the hands, the legs and the body, all belong to the skin image of the baby. Therefore, the embodiments of the present invention can still be achieved even in the case where the face of the baby is covered. Additionally, whether the embodiments of the present invention can be achieved is not related to whether or not the baby has made a sound. Accordingly, the embodiments of the present invention have a better adaptive capability as compared to the prior art.

The skin image of the baby can be converted into a PPG signal, and the needs of the baby are identified based on HRV features calculated from the PPG signal and a HRV feature model established in advance. In other words, in the embodiments of the present invention, it may be deemed that the needs of the baby are identified based on the heart rate variance of the baby, which is the natural physiological and/or psychological reaction of the baby. Under the circumstances, the identification of the baby needs is less likely to be influenced by factors such as the crying, facial expressions and/or actions of the baby. In addition, the misjudgment ratio when identifying the baby needs can be reduced (i.e., the probability of successfully identifying the baby needs can be increased). Accordingly, the embodiments of the present invention have a better identification capability as compared to the prior art.

The heart rate variance of the baby can be obtained by capturing an image with a video camera and then analyzing the image instead of directly measuring the baby with various testing devices. Thus, the embodiments of the present invention have less influence to the baby and are easier to be implemented.

According to the above descriptions, the disclosed example embodiments indeed provide a more effective technology for identifying baby needs.

The detailed technology and preferred embodiments implemented for the present invention are described in the following paragraphs accompanying the appended drawings for people of ordinary skill in the art to well appreciate the features of the claimed invention.

### BRIEF DESCRIPTION OF THE DRAWINGS

FIG. 1 is a schematic view of a system for identifying baby needs in one or more embodiments of the present invention;

FIG. 2 is a schematic view of converting a time-series skin image signal to a PPG signal in one or more embodiments of the present invention;

FIG. 3 is a schematic view of a process for identifying baby needs in one or more embodiments of the present invention; and

FIG. 4 is a flowchart diagram of a method for identifying baby needs in one or more embodiments of the present invention.

### DETAILED DESCRIPTION

In the following description, the present invention will be explained with reference to certain example embodiments thereof. It shall be appreciated that, these example embodiments are not intended to limit the present invention to any particular examples, embodiments, environment, applications or implementations described in these example embodiments. Therefore, description of these example embodiments is only for purpose of illustration rather than to limit the present invention.

In the following embodiments and the attached drawings of the present invention, elements unrelated to the present invention are omitted from depiction; and dimensional relationships among individual elements in the attached drawings are only for ease of understanding, but not to limit the actual scale. Unless stated particularly, same (or similar) element symbols may correspond to same (or similar) elements in the following description.

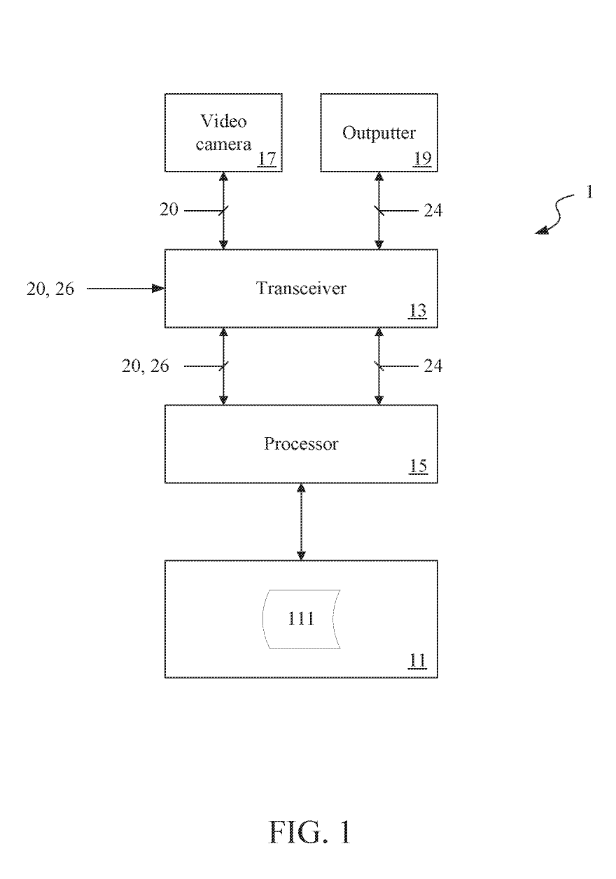

FIG. 1 is a schematic view of a system for identifying baby needs in one or more embodiments of the present invention. Contents shown in FIG. 1 are only for purpose of illustrating embodiments of the present invention instead of limiting the present invention. Referring to FIG. 1, a system 1 for identifying baby needs may generally comprise a storage 11, a processor 15 and a transceiver 13, and the processor 15 may be electrically connected with the storage 11 and the transceiver 13. In some embodiments, in addition to the storage 11, the processor 15 and the transceiver 13, the system 1 for identifying baby needs may additionally comprise a video camera 17 and/or an outputter 19, and the transceiver 13 may be electrically connected to the video camera 17 and the outputter 19 respectively. In some embodiments, the storage 11, the processor 15, the transceiver 13, the video camera 17 and the outputter 19 may be disposed in a same apparatus within the system 1 for identifying baby needs. In some embodiments, the storage 11, the processor 15 and the transceiver 13 may be disposed in a certain apparatus within the system 1 for identifying baby needs, while the video camera 17 or the outputter 19 may be disposed respectively in another apparatus within the system 1 for identifying baby needs. In the case where the video camera 17 or the outputter 19 is disposed in a different apparatus from other elements, the video camera 17 or the outputter 19 may be electrically connected with the transceiver 13 via various wired or wireless ways (for example but not limited to via cables, fibers, Wi-Fi, mobile communication networks or the like). Functions and interactions among the elements will be described hereinafter.

The connection displayed in FIG. 1 above may be direct connection (i.e., connection not via other elements with specific functions) or indirect connection (i.e., connection via other elements with specific functions) depending on different requirements.

The processor 15 may comprise various microprocessors or microcontrollers. The microprocessor or the microcontroller is a kind of programmable specific integrated circuit that is capable of operating, storing, outputting/inputting or the like. Moreover, the microprocessor or the microcontroller can receive and process various coded instructions, thereby performing various logical operations and arithmetical operations and outputting corresponding operation results.

The storage 11 may comprise a primary memory (also called a main memory or an internal memory) for directly connected to the processor 15. The processor 15 can read instruction sets stored in the primary memory and execute these instruction sets if needed. The storage 11 may further comprise a secondary memory (also called an external memory or an auxiliary memory), and the secondary memory connects to the processor 15 via an I/O channel of the memory instead of directly connecting to the processor 15 and uses a data buffer to transmit data to the primary memory. The secondary memory may for example be any of various hard disks, optical disks or the like. The storage 11 may also comprise a third-level memory, i.e., a storage device that can be directly inserted into or pulled out from a computer, e.g., a mobile disk.

The transceiver 13 may comprise various internal connection interfaces (e.g., flat cables of various functions) so that multiple elements disposed in a same apparatus connect and transmit data with each other. In some embodiments, the transceiver 13 may also comprise various input/output interfaces so that multiple elements disposed in different apparatuses connect and transmit data with each other. The input/output interfaces may comprise various wired or wireless communication interfaces (which are for example but not limited to: a cable interface, a fiber interface, a Wi-Fi interface, a mobile communication network interface or the like).

The video camera 17 may include various photographic devices capable of capturing image signals. The outputter 19 may include apparatuses capable of outputting various kinds of data (e.g., image data, sound data or the like), which are for example but not limited to a screen, a touch screen, a projector, a mobile phone, a notebook computer, a tablet computer, a loudspeaker or the like.

Still referring to FIG. 1, the storage 11 may store a heart rate variability (HRV) feature model 111, and the HRV feature model 111 may comprise a relationship between HRV features and baby needs. Specifically, different baby needs (e.g., need to be soothed, being hungry, feeling uncomfortable (e.g., need to stool or pee) or the like) have different heart rate variances, and different heart rate variances will be reflected by different HRV features, so there is a relationship between the HRV features and the baby needs. In some embodiments, the HRV feature model 111 stored in the storage 11 may be constructed by the processor 15 (which will be detailed later). In some embodiments, the HRV feature model 111 stored in the storage 11 may also be an HRV feature model that has been constructed externally.

The transceiver 13 may be configured to receive a time-series skin image signal 20 of any baby, and transmit the time-series skin image signal 20 to the processor 15. The time-series skin image signal 20 comprises an image or a picture of the skin covering at least a portion of the body (e.g., the face, hands or feet or the like) of the baby. For example, the time-series skin image signal 20 may be obtained by capturing an image of the baby with the video camera 17. In some embodiments, the video camera 17 may be a general video camera, a photo camera, or an infrared camera, and an advantage of the infrared camera is that the time-series skin image signal 20 can still be obtained by capturing the baby even at night or in the case without sufficient light. As another example, the time-series skin image signal 20 may also be a time-series skin image signal of the baby inputted to the transceiver 13 by the user itself via a user interface.

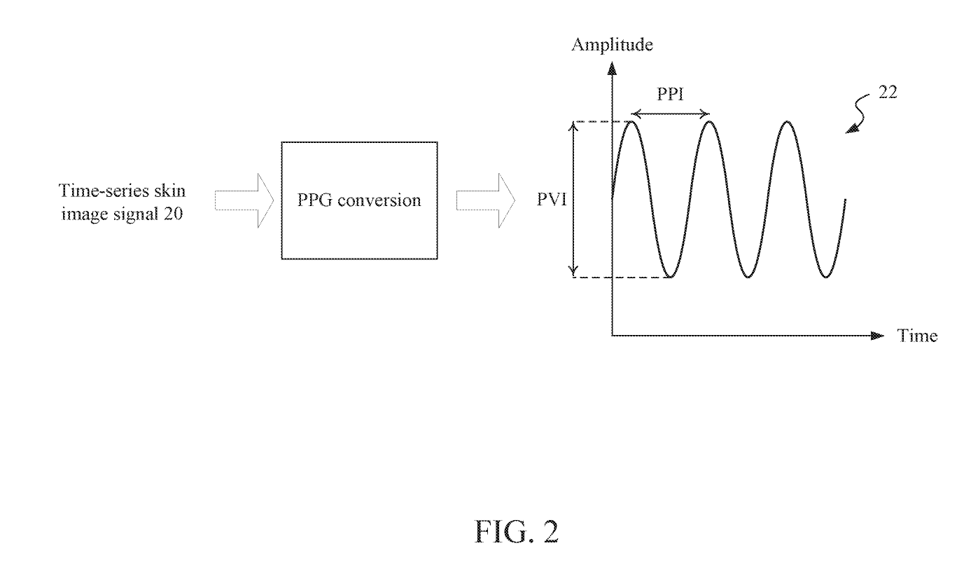

FIG. 2 is a schematic view of converting a time-series skin image signal to a PPG signal in one or more embodiments of the present invention. Contents shown in FIG. 2 are only for purpose of illustrating embodiments of the present invention instead of limiting the present invention. Referring to FIG. 1 and FIG. 2, the processor 15 may be configured to convert the time-series skin image signal 20 to a target PPG signal 22.

For example, in order to convent the time-series skin image signal 20 to the target PPG signal 22, the processor 15 may substantially perform the following operations: (1) a detrend smoothing calculation, i.e., detrending by removing the mean value or the linear trend through vectors or matrixes; (2) five-point moving average filter smoothing, i.e., by calculating a simple moving average, an exponential moving average, a triangular moving average, a weighted moving average and a modified moving average of a time sequence target of a vector or data; (3) bandpass filter filtering, i.e., attenuating frequencies beyond a particular range of frequency and retaining frequencies within a particular range of frequency, and (4) a blood vessel pulse peak (BVP peak) searching algorithm, i.e., searching for a vector having local maxima of an input signal vector.

In some embodiments, the aforesaid operations may be eliminated to some extent depending on needs. In some embodiments, in addition to the aforesaid operations, the processor 15 may further perform other operations which are for example but not limited to: noise separating, noise filtering, interpolating and re-sampling, the Fast Fourier Transform or the like. Reference may also be made to “Remote measurement of cognitive stress via heart rate variability” (36th Annual International Conference of the IEEE Engineering in Medicine and Biology Society, 2014, pp. 2957-2960) written by D. McDuff or “A survey of remote optical photoplethysmographic imaging methods” (37th Annual International Conference of the IEEE Engineering in Medicine and Biology Society (EMBC), 2015, pp. 6398-6404) written by D. J. McDuff for how to convert the time-series skin image signal 20 to the target PPG signal 22, and the two documents are incorporated herein by reference in their entirety.

Still referring to FIG. 1 and FIG. 2, after obtaining the target PPG signal 22, the processor 15 may be further configured to calculate a set of target HRV features according to the target PPG signal 22 and identify a target need of the baby according to the HRV feature model 111 and the set of target HRV features. In some embodiments, after identifying the target need of the baby, the processor 15 may further transmit information 24 relevant to the target need of the baby to the outputter 19 via the transceiver 13, and the outputter 19 may provide the information 24 to the user through images and/or sounds.

Depending on different needs, the set of target HRV features calculated by the processor 15 according to the target PPG signal 22 may comprise one or more HRV features. For example, referring to FIG. 2, the set of target HRV features may comprise a feature relevant to a peak-to-peak interval (PPI) sequence and a feature relevant to a peak-to-valley interval (PVI) sequence, wherein the PPI sequence refers to the time difference between peaks within a time period of the target PPG signal 22, and the PVI sequence refers to the amplitude difference between each peak and valley within a time period of the target PPG signal 22. The set of target HRV features may further comprise other HRV features calculated according to the target PPG signal 22 without being limited to the aforesaid HRV features, and the other HRV features are for example but not limited to: time domain features, respiratory frequency features and waveform features or the like.

In some embodiments, in order to reduce the calculating amount or increase the calculating efficiency, the processor 15 may also first select at least one primary target HRV feature from the set of target HRV features, and then identify the target need of the baby only based on the HRV feature model 111 and the at least one primary target HRV feature.

For example, in some embodiments, if a certain baby need has a larger relevance to the waveform variation, the amplitude variation and the regularity of the waveform of the target PPG signal 22 as compared to other factors, then the processor 15 may select the following primary target HRV features from the set of target HRV features: a target peak-to-peak interval (PPI) feature, a target peak-to-valley interval (PVI) feature and a target PPI standard deviation feature. The target PPI feature may correspond to a target PPI variation within a target time period (e.g., 1 minute, 5 minutes, 10 minutes or 20 minutes or the like), so it can reflect the waveform variation (the frequency variation) of the target PPG signal 22. The target PVI feature may correspond to a target PVI variation within the target time period, so it can reflect the amplitude variation of the target PPG signal 22. The target PPI standard deviation feature may correspond to a standard deviation of the target PPI variation, so it can reflect the regularity of the waveform of the target PPG signal 22.

For ease of illustration, the target PPI feature may be represented as:

f(PPI)=ax+b (1)

where x is a sampling number, a is a slope, and b is a constant.

For ease of illustration, the target PVI feature may be represented as:

f(PVI)=cy+d (2)

where y is a sampling number, c is a slope, and d is a constant.

For ease of illustration, the target PPI standard deviation feature may be represented as:

$$SDNN = \sqrt{\frac{{\sum }_{i = 1}^{n} {( {R}_{i} - {R}_{m} )}^{2}}{n}} \qquad ( 3 )$$

where Rᵢ is the i^{th} PPI, Rₘ is an average of PPI, and n is the number of PPI.

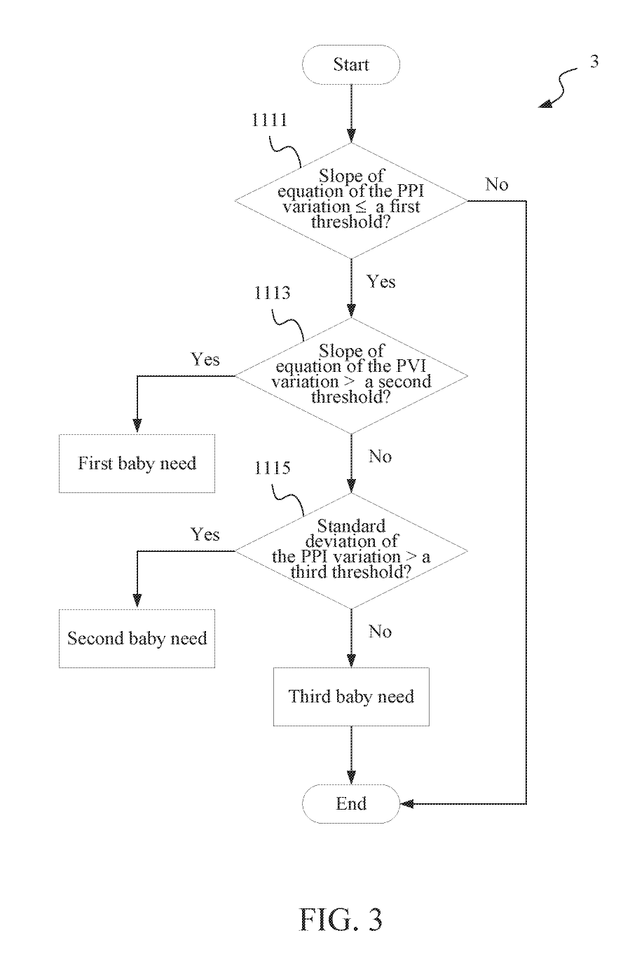

FIG. 3 is a schematic view of a process 3 for identifying baby needs in one or more embodiments of the present invention. Contents shown in FIG. 3 are only for the purpose of illustrating the embodiments of the present invention instead of limiting the present invention. Referring to FIG. 1 to FIG. 3, the processor 15 may identify the target need of the baby based on the HRV feature model 111 and the target PPI feature, the PVI feature, and the target PPI standard deviation feature. Specifically, in a determining step 1111 of the process 3 for identifying baby needs, the processor 15 may determine whether a slope of equation of the PPI variation (i.e., the slope a in the equation (1)) is less than or equal to a first threshold. If the result of the determining step 1111 is no, then the processor 15 may determine that the baby has no need currently and may end the identifying process. If the result of the determining step 1111 is yes, then the process may enter into another determining step 1113 of the process 3 for identifying baby needs, in which the processor 15 further determines whether a slope of equation of the PVI variation (i.e., the slope c in the equation (2)) is greater than a second threshold. If the result of the determining step 1113 is yes, then the processor 15 may identify the target need of the baby as a first baby need. If the result of the determining step 1113 is no, then the process may enter into the next determining step 1115 of the process 3 for identifying baby needs, in which the processor 15 further determines whether a standard deviation of the PPI variation (i.e., the SDNN in the equation (3)) is greater than a third threshold. If the result of the determining step 1115 is yes, then the processor 15 identifies the target need of the baby as a second baby need. If the result of the determining step 1115 is no, then the processor 15 identifies the target need of the baby as a third baby need. In some embodiments, the order in which the determining steps 1111, 1113 and 1115 are executed may be adjusted arbitrarily instead of being limited to the order shown in FIG. 3.

The first threshold, the second threshold, the third threshold, the first baby need, the second baby need and the third baby need may be decided and adjusted according to analysis, experiments and measurement performed in advance for needs of multiple babies. For example, in some embodiments, the first baby need, the second baby need and the third baby need may be respectively “need to be soothed”, “being hungry” and “feeling uncomfortable” in the case where the first threshold, the second threshold and the third threshold are respectively “about lager than 0”, “about lager than 0” and “0.5”.

In some embodiments, in addition to the determining steps 1111, 1113 and 1115, the process 3 for identifying baby needs may further comprise more other determining steps to identify more kinds of baby needs, and the number of the determining steps depends on the number of HRV features calculated by the processor 15.

In some embodiments, the HRV feature model 111 may be constructed by the processor 15. In detail, referring to FIG. 1 to FIG. 3, the transceiver 13 may be configured to receive a plurality of reference PPG signals 26, and transmit the plurality of reference PPG signals 26 to the processor 15. For example, the plurality of reference PPG signals 26 may be a plurality of PPG signals obtained in advance by directly measuring one or more babies using various physiological signal measuring instruments, and each of the plurality of PPG signals may be a signal measured when a baby generates a certain need (i.e., each of the plurality of reference PPG signals 26 may respectively correspond to a baby need). A plurality of reference PPG signals 26 may correspond to a same baby need.

The processor 15 may be further configured to calculate a plurality of sets of reference HRV features according to the plurality of reference PPG signals 26 and select at least one reference HRV feature from each of the plurality of sets of reference HRV features. The at least one reference HRV feature is for example but not limited to: a reference PPI feature, a reference PVI feature and a reference PPI standard deviation feature. The reference PPI feature and the reference PVI feature may correspond to a reference PPI variation and a reference PVI variation within a reference time period (e.g., 1 minute, 5 minutes, 10 minutes or 20 minutes or the like) respectively, and the reference PPI standard deviation feature may correspond to a standard deviation of the reference PPI variation. The reference time period may be the same as or different from the target time period described above.

In detail, the processor 15 may select the reference PPI feature, the reference PVI feature, and the reference PPI standard deviation feature from each of the plurality of sets of reference HRV features according to an optimization algorithm. For example, the optimization algorithm may comprise a sequential backward selection (SBS) algorithm and a genetic algorithm. The SBS algorithm works starting with the full feature set and performing the search until the desired feature number is reached. The genetic algorithm encodes the selected features into a gene, and then generates and searches for a classification decision-making tree of a high accuracy through mating and mutation, thereby observing the identification result of the reserved features and continuously calculating the feature combination converged to the highest resolution (e.g., the reference PPI feature, the reference PVI feature and the reference PPI standard deviation feature described previously). Reference may be made to “Emotion state identification based on heart rate variability and genetic algorithm” (in 2015 37th Annual International Conference of the IEEE Engineering in Medicine and Biology Society (EMBC), 2015, pp. 538-541) written by Sung-Nien Yu for details thereof, and this document is incorporated herein by reference in its entirety.

The processor 15 may be further configured to define the first threshold according to a plurality of slopes of equations of the plurality of reference PPI variations, define the second threshold according to a plurality of slopes of equations of the plurality of reference PVI variations, and define the third threshold according to the plurality of standard deviations of the plurality of reference PPI variations.

For example, if the transceiver 13 has received six hundred reference PPG signals 26, then the processor 15 may calculate six hundred reference PPI features for the six hundred reference PPG signals 26 to form a new PPI time sequence, and calculate the time sequence into a first linear equation (e.g., the equation (1)) to obtain a slope of the first linear equation. The processor 15 may also calculate six hundred reference PVI features for the six hundred reference PPG signals 26 to form a new PVI time sequence, and calculate the time sequence into a second linear equation (e.g., the equation (2)) to obtain a slope of the second linear equation. The processor 15 may further calculate six hundred reference PPI standard deviation features for the six hundred reference PPI features according to the equation (3). Then, the processor 15 may define the first threshold according to the slope of the first linear equation, define the second threshold according to the slope of the second linear equation, and define the third threshold according to the reference PPI standard deviation features after being averaged. Finally, the processor 15 may define the first baby need, the second baby need and the third baby need according to the first threshold, the second threshold and the third threshold to establish the HRV feature model 111. The first threshold, the second threshold, the third threshold, the first baby need, the second baby need and the third baby need form a relationship between the HRV features and the baby needs.

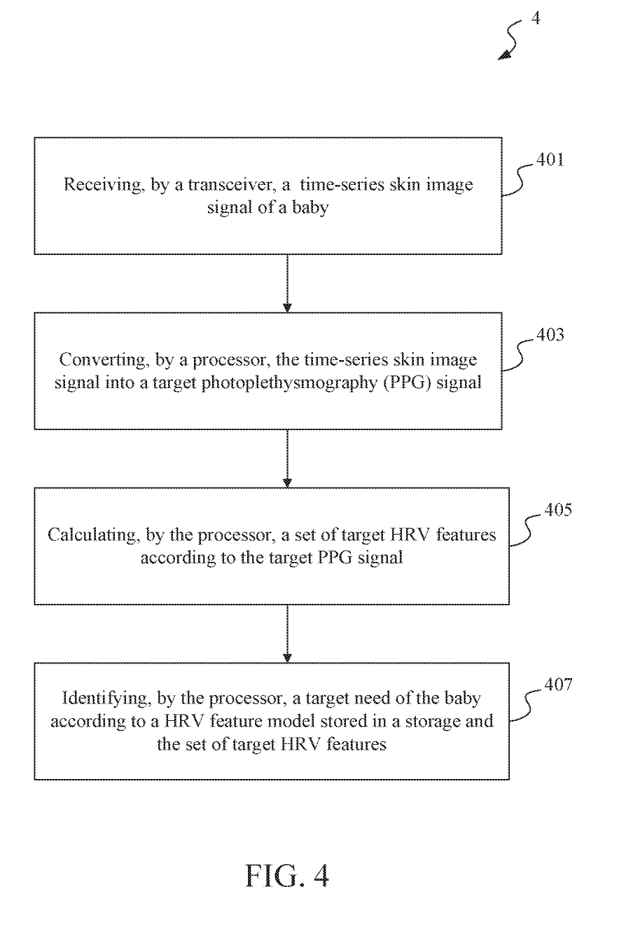

FIG. 4 is a schematic view of a method for identifying baby needs in one or more embodiments of the present invention. Contents shown in FIG. 4 are only for purpose of illustrating embodiments of the present invention instead of limiting the present invention. Referring to FIG. 4, a method 4 for identifying baby needs may comprise the following steps: receiving, by a transceiver, a time-series skin image signal of a baby (labeled as 401); converting, by a processor, the time-series skin image signal into a target PPG signal (labeled as 403); calculating, by the processor, a set of target HRV features according to the target PPG signal (labeled as 405); and identifying, by the processor, a target need of the baby according to a HRV feature model stored in a storage and the set of target HRV features (labeled as 407), wherein the HRV feature model comprises a relationship between HRV features and baby needs.

In some embodiments, the method 4 for identifying baby needs may further comprise the following steps: selecting, by the processor, at least one primary target HRV feature from the set of target HRV features; wherein the step of identifying the target need of the baby is: identifying, by the processor, the target need of the baby according to the HRV feature model and the at least one primary target HRV feature.

In some embodiments, the at least one primary target HRV feature may include a target peak-to-peak interval (PPI) feature, a target peak-to-valley interval (PVI) feature and a target PPI standard deviation feature, the target PPI feature and the target PVI feature correspond to a target PPI variation and a target PVI variation within a target time period respectively, and the target PPI standard deviation feature corresponds to a standard deviation of the target PPI variation.

In some embodiments, the step 407 may further comprise the following steps: identifying, by the processor, the target need of the baby as a first baby need when a slope of equation of the target PPI variation is less than or equal to a first threshold and a slope of equation of the target PVI variation is greater than a second threshold; identifying, by the processor, the target need of the baby as a second baby need when the slope of equation of the target PPI variation is less than or equal to the first threshold, the slope of equation of the target PVI variation is less than or equal to the second threshold and the standard deviation of the target PPI variation is greater than a third threshold; and identifying, by the processor, the target need of the baby as a third baby need when the slope of equation of the target PPI variation is less than or equal to the first threshold, the slope of equation of the target PVI variation is less than or equal to the second threshold and the standard deviation of the target PPI variation is less than or equal to the third threshold.

In some embodiments, the HRV feature model may further comprise a first threshold, a second threshold and a third threshold, and the method 4 for identifying baby needs may further comprise the following steps: receiving, by the transceiver, a plurality of reference PPG signals; calculating, by the processor, a plurality of sets of reference HRV features according to the plurality of reference PPG signals and selecting, by the processor, a reference PPI feature, a reference PVI feature and a reference PPI standard deviation feature from each of the plurality of sets of reference HRV features, wherein the reference PPI feature and the reference PVI feature correspond to a reference PPI variation and a reference PVI variation within a reference time period respectively, and the reference PPI standard deviation feature corresponds to a standard deviation of the reference PPI variation; and defining, by the processor, the first threshold according to a plurality of slopes of equations of the plurality of reference PPI variations, defining, by the processor, the second threshold according to a plurality of slopes of equations of the plurality of reference PVI variations, and defining, by the processor, the third threshold according to the plurality of standard deviations of the plurality of reference PPI variations.

In some embodiments, the processor may select the reference PPI feature, the reference PVI feature, and the reference PPI standard deviation feature from each of the plurality of sets of reference HRV features according to an optimization algorithm.

In some embodiments, each of the plurality of reference PPG signals may correspond to a baby need respectively, and the relationship comprised in the HRV feature model may be established according to the reference PPI features, the reference PVI features and the reference PPI standard deviation features of the plurality of reference PPG signals as well as the baby needs corresponding to the plurality of reference PPG signals.

In some embodiments, the method 4 for identifying baby needs may further comprise the following step: providing the time-series skin image signal by a video camera.

In some embodiments, the video camera may be an infrared video camera.

In some embodiments, the method 4 for identifying baby needs may further comprise the following step: outputting information related to the target need of the baby by an outputter.

In some embodiments, the method 4 for identifying baby needs may be applied to the system 1 for identifying baby needs, and may perform all the corresponding steps for implementing the system 1 for identifying baby needs. All the corresponding steps of the method 4 for identifying baby needs can be appreciated directly and unambiguously by people of ordinary skill in the art based on the above description of the system 1 for identifying baby needs, and thus will not be further described herein.

The above disclosure is related to the detailed technical contents and inventive features thereof. People of ordinary skill in the art may proceed with a variety of modifications and replacements based on the disclosures and suggestions of the invention as described without departing from the characteristics thereof. Nevertheless, although such modifications and replacements are not fully disclosed in the above descriptions, they have substantially been covered in the following claims as appended.

What is claimed is:

1. A system for identifying baby needs, comprising:
a storage, being configured to store a heart rate variability (HRV) feature model that comprises a relationship between HRV features and baby needs;
a transceiver, being configured to receive a time-series skin image signal of a baby; and
a processor electrically connected to the storage and the transceiver, being configured to:
convert the time-series skin image signal into a target photoplethysmography (PPG) signal;
calculate a set of target HRV features according to the target PPG signal; and
identify a target need of the baby according to the HRV feature model and the set of target HRV features.

2. The system of claim 1, wherein:
the processor further selects at least one primary target HRV feature from the set of target HRV features, and identifies the target need of the baby according to the HRV feature model and the at least one primary target HRV feature.

3. The system of claim 2, wherein:
the at least one primary target HRV feature includes a target peak-to-peak interval (PPI) feature, a target peak-to-valley interval (PVI) feature and a target PPI standard deviation feature, the target PPI feature and the target PVI feature correspond to a target PPI variation and a target PVI variation within a target time period respectively, and the target PPI standard deviation feature corresponds to a standard deviation of the target PPI variation.

4. The system of claim 3, wherein:
the processor identifies the target need of the baby as a first baby need when a slope of equation of the target PPI variation is less than or equal to a first threshold and a slope of equation of the target PVI variation is greater than a second threshold;
the processor identifies the target need of the baby as a second baby need when the slope of equation of the target PPI variation is less than or equal to the first threshold, the slope of equation of the target PVI variation is less than or equal to the second threshold and the standard deviation of the target PPI variation is greater than a third threshold; and
the processor identifies the target need of the baby as a third baby need when the slope of equation of the target PPI variation is less than or equal to the first threshold, the slope of equation of the target PVI variation is less than or equal to the second threshold and the standard deviation of the target PPI variation is less than or equal to the third threshold.

5. The system of claim 1, wherein:
the HRV feature model further comprises a first threshold, a second threshold and a third threshold;
the transceiver is further configured to receive a plurality of reference PPG signals;
the processor is further configured to calculate a plurality of sets of reference HRV features according to the plurality of reference PPG signals and select a reference PPI feature, a reference PVI feature and a reference PPI standard deviation feature from each of the plurality of sets of reference HRV features, the reference PPI feature and the reference PVI feature correspond to a reference PPI variation and a reference PVI variation within a reference time period respectively, and the reference PPI standard deviation feature corresponds to a standard deviation of the reference PPI variation; and
the processor defines the first threshold according to a plurality of slopes of equations of the plurality of reference PPI variations, defines the second threshold according to a plurality of slopes of equations of the plurality of reference PVI variations, and defines the third threshold according to the plurality of standard deviations of the plurality of reference PPI variations.

6. The system of claim 5, wherein the processor selects the reference PPI feature, the reference PVI feature, and the reference PPI standard deviation feature from each of the plurality of sets of reference HRV features according to an optimization algorithm.

7. The system of claim 5, wherein each of the plurality of reference PPG signals corresponds to a baby need respectively, and the relationship comprised in the HRV feature model is established according to the reference PPI features, the reference PVI features and the reference PPI standard deviation features of the plurality of reference PPG signals as well as the baby needs corresponding to the plurality of reference PPG signals.

8. The system of claim 1, further comprising a video camera, wherein the video camera is electrically connected to the transceiver and is configured to provide the time-series skin image signal.

9. The system of claim 8, wherein the video camera is an infrared video camera.

10. The system of claim 1, further comprising an outputter, wherein the outputter is electrically connected to the transceiver and is configured to output information related to the target need of the baby.

11. A method for identifying baby needs, comprising:
receiving, by a transceiver, a time-series skin image signal of a baby;
converting, by a processor, the time-series skin image signal into a target photoplethysmography (PPG) signal;
calculating, by the processor, a set of target HRV features according to the target PPG signal; and
identifying, by the processor, a target need of the baby according to a HRV feature model stored in a storage and the set of target HRV features, wherein the HRV feature model comprises a relationship between HRV features and baby needs.

12. The method of claim 11, further comprising:
selecting, by the processor, at least one primary target HRV feature from the set of target HRV features;
wherein the step of identifying the target need of the baby is: identifying, by the processor, the target need of the baby according to the HRV feature model and the at least one primary target HRV feature.

13. The method of claim 12, wherein the at least one primary target HRV feature includes a target peak-to-peak interval (PPI) feature, a target peak-to-valley interval (PVI) feature and a target PPI standard deviation feature, the target PPI feature and the target PVI feature correspond to a target PPI variation and a target PVI variation within a target time period respectively, and the target PPI standard deviation feature corresponds to a standard deviation of the target PPI variation.

14. The method of claim 13, further comprising:
identifying, by the processor, the target need of the baby as a first baby need when a slope of equation of the target PPI variation is less than or equal to a first threshold and a slope of equation of the target PVI variation is greater than a second threshold;
identifying, by the processor, the target need of the baby as a second baby need when the slope of equation of the target PPI variation is less than or equal to the first threshold, the slope of equation of the target PVI variation is less than or equal to the second threshold and the standard deviation of the target PPI variation is greater than a third threshold; and
identifying, by the processor, the target need of the baby as a third baby need when the slope of equation of the target PPI variation is less than or equal to the first threshold, the slope of equation of the target PVI variation is less than or equal to the second threshold and the standard deviation of the target PPI variation is less than or equal to the third threshold.

15. The method of claim 11, wherein the HRV feature model further comprises a first threshold, a second threshold and a third threshold, and the method further comprising:
receiving, by the transceiver, a plurality of reference PPG signals;
calculating, by the processor, a plurality of sets of reference HRV features according to the plurality of reference PPG signals and selecting, by the processor, a reference PPI feature, a reference PVI feature and a reference PPI standard deviation feature from each of the plurality of sets of reference HRV features, the reference PPI feature and the reference PVI feature corresponding to a reference PPI variation and a reference PVI variation within a reference time period respectively, and the reference PPI standard deviation feature corresponding to a standard deviation of the reference PPI variation; and
defining, by the processor, the first threshold according to a plurality of slopes of equations of the plurality of reference PPI variations, defining, by the processor, the second threshold according to a plurality of slopes of equations of the plurality of reference PVI variations, and defining, by the processor, the third threshold according to the plurality of standard deviations of the plurality of reference PPI variations.

16. The method of claim 15, wherein the processor selects the reference PPI feature, the reference PVI feature, and the reference PPI standard deviation feature from each of the plurality of sets of reference HRV features according to an optimization algorithm.

17. The method of claim 15, wherein each of the plurality of reference PPG signals corresponds to a baby need respectively, and the relationship comprised in the HRV feature model is established according to the reference PPI features, the reference PVI features and the reference PPI standard deviation features of the plurality of reference PPG signals as well as the baby needs corresponding to the plurality of reference PPG signals.

18. The method of claim 11, further comprising:
providing, by a video camera, the time-series skin image signal.

19. The method of claim 18, wherein the video camera is an infrared video camera.

20. The method of claim 11, further comprising:
outputting, by an outputter, information related to the target need of the baby.
